(19) United States
(12) Patent Application Publication
(10) Pub. No.: US 2006/0210612 A1
(43) Pub. Date: Sep. 21, 2006

(54) Make-up-removing article

(75) Inventors: Pascal Simon, Thiais (FR); Josselyn Simon, Thiais (FR)

Correspondence Address: C. IRVIN MCCLELLAND;OBLON, SPIVAK, MCCLELLAND, MAIER & NEUSTADT, P.C., 1940 DUKE STREET, ALEXANDRIA, VA, 22314, US

(73) Assignee: L'OREAL, Paris (FR)

(21) Appl. No.: 11/341,530

(22) Filed: Jan. 30, 2006

Related U.S. Application Data
(60) Provisional application No. 60/654,457, filed on Feb. 22, 2005.

(30) Foreign Application Priority Data
Feb. 9, 2005 (FR) 05 50386

(51) Int. Cl. A61K 9/70

(52) U.S. Cl. 424/443

(57) ABSTRACT

An article containing:(A) a water-insoluble hydrophilic polyurethane foam support, and, impregnated on the support,(B) a composition containing separate, non-emulsified aqueous and oily phases, the aqueous and oily phases being present in a ratio by weight ranging from 25/75 to 90/10 (aqueous/oily), the oily phase having a melting point of less than 25° C. The foam can in particular be an open cell polyurethane foam. This article can be used in particular for cleansing and/or removing make-up from the skin of the face and/or body as well as for removing make-up from the eyes.

### REFERENCE TO PRIOR APPLICATIONS

This application claims priority to U.S. provisional application 60/654,457 filed Feb. 22, 2005, and to French patent application 0550386 filed Feb. 9, 2005, both incorporated herein by reference.

### FIELD OF THE INVENTION

The invention relates to an article comprising at least one water-insoluble support and a cosmetic composition comprising two separate immiscible phases, and also to the uses of the article, for example in the cosmetics field, in particular for cleansing and/or removing make-up from human skin, more especially the skin of the face, lips and/or eyes.

Additional advantages and other features of the present invention will be set forth in part in the description that follows and in part will become apparent to those having ordinary skill in the art upon examination of the following or may be learned from the practice of the present invention. The advantages of the present invention may be realized and obtained as particularly pointed out in the appended claims. As will be realized, the present invention is capable of other and different embodiments, and its several details are capable of modifications in various obvious respects, all without departing from the present invention. The description is to be regarded as illustrative in nature, and not as restrictive.

### BACKGROUND OF THE INVENTION

Articles such as cosmetic cleansing and make-up-removing wipes are commonly used and are valued for their practical side as they are disposable, they are impregnated with the necessary and sufficient amount of cleansing or make-up-removing product and they avoid the handling and the transportation of bottles containing lotions or milks. These articles are generally composed of a support made of a material of natural or synthetic origin which is preferably a nonwoven, the support being impregnated with a composition suitable for the desired purpose, for example the cleansing or removal of make-up from the skin, or caring for the skin.

Moreover, it is known to use, as make-up-removing composition, compositions having two separate phases, an aqueous phase and an immiscible oily phase, as is disclosed, for example, in the documents FR-A-2 847 468, FR-A-2 753 090 and EP-A-0 370 856. The compositions of this type, composed of two separate phases, in particular of an aqueous phase and of an oily phase, which are separate and nonemulsified with one another on standing, are generally denoted under the term of “two-phase composition”. They are distinguished from emulsions in that, on standing the two phases are separate instead of being emulsified with one another and that they form an O/W dispersion only when they are agitated. The use of these two-phase compositions thus requires preliminary agitation in order to form a dispersion at the time of use, the latter having to be of sufficient quality and of sufficient stability to allow homogeneous application of the two phases on the skin or any other keratinous substance on which it is applied. On standing, the phases have to rapidly separate and recover their initial state, this phenomenon being better known under the term of “phase separation”.

This type of make-up-remover makes it possible to remove all types of waterproof or nonwaterproof make-up and it has the advantage of making possible removal of make-up which is as effective as with an oil while having advantageous cosmetic properties, in contrast to the oils, which, for their part, are troublesome to use. This is because the compositions composed solely of oils leave a greasy residual film on the skin, whereas a two-phase composition, once agitated, is converted into a dispersion of droplets of oil in the aqueous phase and does not leave a greasy residual film, while retaining a make-up-removing effectiveness comparable to that of a composition comprising only oil.

With the aim of combining the practical side of the wipes and the effectiveness of the make-up-removers comprising two separate phases, it appeared judicious to wish to impregnate this type of make-up-remover on a nonwoven. The impregnation of the two-phase compositions on a nonwoven did not present problems in so far as the viscosity of the composition is low. Moreover, the impregnated product is released very well, which makes possible good removal of make-up. However, the stability over time of such a product is unsatisfactory as, in packagings in which a stack of make-up-removing wipes or pads is assembled, a phenomenon of sedimentation is observed with the deposition of the aqueous phase at the bottom of the box or of the wrapping comprising the wipes, with the result that, after a relatively short storage time of one to two weeks, the wipes situated at the top of the stack are more impregnated with oil than those at the bottom and, for this reason, each wipe is no longer impregnated with the same proportion of each phase according to its location in the stack, as emerges from the tests presented later.

Thus, the need remains to have available an article of wipe type comprising a composition in which effectiveness and comfort are combined, as is the two-phase composition, which remains stable in composition, so that several articles stacked in a packet remain impregnated in the same way over time.

### SUMMARY OF THE INVENTION

The inventors have found, surprisingly, that the use of a support made of hydrophilic polyurethane foam makes it possible to prevent the above-described phenomenon of sedimentation while retaining the good cosmetic properties of nonwoven wipes (softness, ready availability of the impregnation composition for wiping) and of the two-phase composition.

### BRIEF DESCRIPTION OF THE DRAWINGS

FIG. 1 reflects the proportions of aqueous phase and of oily phase over time of a comparative nonwoven support.

FIG. 2 reflects the proportions of aqueous and of oily phase over time of a support of the present invention.

### DETAILED DESCRIPTION OF THE PREFERRED EMBODIMENTS

One subject-matter of the present invention is thus an article comprising (A) a water-insoluble support comprised of a hydrophilic polyurethane foam and (B) a cosmetic composition impregnated on the support and comprising a separate aqueous phase and a separate oily phase in a ratio by weight ranging from 25/75 to 90/10, the constituents of the oily phase, and the oily phase itself, having a melting point of less than 25° C.

The hydrophilic polyurethane foam is formed before impregnation with the cosmetic composition, which means that the cosmetic composition is thus not impregnated in situ during the manufacture of the foam, as is the case, for example, for the emulsion disclosed in the document U.S. Pat. No. 4,806,572, as such an in situ impregnation exhibits the disadvantage of making it necessary to have an effective amount of emulsifying surfactant and of not making possible the impregnation of a two-phase composition.

Furthermore, in the present patent application, the aqueous phase and the oily phase are impregnated on the support simultaneously or separately and the polyurethane support is preferably the only impregnation support.

The document WO-A-2004/006879 discloses a hygiene product which comprises an applicator on which a lipid phase and an aqueous phase have been applied. However, the lipid phase is solid or semisolid at ambient temperature and it generally comprises a wax, which would be completely out of the question in the present invention as the presence of a compound which is solid or semisolid at ambient temperature in the composition claimed according to the present invention would greatly reduce, indeed even nullify, the make-up-removing effectiveness of the article as these compounds, due to their solid to semisolid state, are not effective solvents for dissolving and removing the oily emulsions and other waxy compositions of which make-up products are composed. In addition, in this document, the use of a polyurethane foam is limited to the impregnation of a lipid phase, the aqueous phase being impregnated on a cellulose sponge.

The article claimed in the present patent application exhibits the advantage of having an impregnation composition which remains stable and identical, even if there is a stack of articles, as is shown by the results presented with the examples.

In addition, the article according to the invention exhibits the advantage of being very easy to handle as it is used directly on the skin without having to wet it and it can be applied by simple wiping over the skin, like a make-up-removing wipe or a make-up-removing cotton pad.

While not bound by a particular theory, it is believed that during application to the skin, the pressure exerted by the hand of the user on the article during wiping causes the composition to pass from the inside of the foam towards the outside and the passage of the two-phase composition through the cells of the foam causes a shearing effect which converts it into a fine dispersion of droplets of oil in the aqueous phase.

The article according to the invention is in particular a cosmetic article appropriate for cleansing or removing make-up from the skin of the face and/or of the body, the lips and/or the eyes. It can in particular constitute, for example, a wipe, compress, sponge or pad. It can have any shape suitable for practical use on the face or body.

Another subject-matter of the invention is the cosmetic use of the article as defined above for cleansing and/or removing make-up from the skin, lips and/or eyes.

A further subject-matter of the invention is a cosmetic process for cleansing and/or removing make-up from the skin, lips and/or eyes which comprises passing, over the skin, lips and/or eyes, an article as defined above.

As the composition used according to the invention for the impregnation of the support is preferably intended for topical application, it preferably comprises a physiologically acceptable medium, that is to say a medium compatible with the skin, mucous membranes (lips), hair and scalp.

Support

The support is preferably a hydrophilic polyurethane foam. The term “hydrophilic” is understood to mean a foam which absorbs water. Preferably, the foam used according to the invention has a maximum water-absorbing power of 0.4 to 3.5 g/cm³ and a water-retaining power of 0.07 to 2 g/cm³.

Water-absorbing power corresponds to the maximum amount of water (in grams) which can be absorbed with regard to a volume of dry foam (in cm³). This amount is determined by weighing a foam with a predetermined volume before impregnation, by then immersing the foam in water, the foam thus being saturated with water when removed, and by weighing the impregnated foam. It is also possible to simply determine the volume of water before and after immersion of the foam, the difference corresponding to the weight of water absorbed by the volume of foam.

The water-retaining power corresponds to the amount of water (in grams) which remains in the foam with respect to the volume of dry foam (in cm³) after application to the foam, impregnated with its maximum impregnation capacity, of a force of 80 newtons for one second.

Furthermore, this foam is preferably elastic. The term “elastic” is understood to mean a foam having an elasticity ranging from 10% to 500% in elongation, preferably from 50% to 150% in elongation.

The support is preferably made of polyurethane foam, as disclosed, for example, in the document DE10327707. These are open cell and preferably fine pore polyurethane foams. The polyurethane foam can be obtained according to conventional techniques for the preparation of polyurethanes, for example by preparation of a polyurethane prepolymer having free isocyanate groups, this prepolymer being obtained from a diisocyanate and from a polyol or from a polyester or from a polyether polyol, followed by hydrolysis of the prepolymer on contact with water or reaction with an amine compound, in the presence of appropriate catalysts. Use may also be made of any other preparation process known to a person skilled in the art.

The foam constituting the support can have any thickness, for example ranging from 0.5 to 10 mm and better still from 1 to 5 mm.

In addition, this support can have any size and any shape appropriate for the desired purpose. It can, for example, be rectangular, round, square or oval in shape. It generally has a surface area of between 0.005 m² and 0.1 m², preferably between 0.01 m² and 0.05 m².

Use may be made, as foam, for example, of the foam sold by Dicon, with a thickness of 3 mm, or that sold by Martini, with a thickness of 3 mm, or that sold by Otto Bock under then name PU-Schwamm, with a thickness of 5 mm, or that sold by Rynel Medical Foam, with a thickness of 5 mm.

The degree of impregnation of the composition on the support is not limited and generally ranges from 10 to 1500% by weight, with respect to the weight of support, preferably from 50 to 1000% and better still between 70 and 500%.

The techniques for the impregnation of the supports with the compositions used according to the invention are well known in this field and are all applicable to the present invention. Thus, the two-phase composition can be added to the support, for example, by immersion, coating, spraying, and the like. It can be prepared first in the two-phase form by stirring the oily phase and the aqueous phase and then impregnated on the support, or else one of the phases and then the other phase can be added to the support one after the other, the order of introduction of the phases not being determining. According to a preferred embodiment of the invention, the support is impregnated by simultaneous introduction of the two phases of the two-phase composition with stirring, either via a single inlet, to ensure homogeneous impregnation of the two phases, or simultaneously but independently via two separate inlets.

Two-phase Composition

The impregnation composition according to the invention comprises a separate aqueous phase and a separate oily phase. These two phases are separate, that is to say that they are visible the one above the other on standing and form a dispersion only after agitation of the mixture and that they separate into two phases when agitation ceases. In this regard the invention composition comprises separate, non-emulsified aqueous and oily phases. This description is applicable to the composition prior to impregnation on the support. Of course, since the support is typically a sponge or foam or wipe, the composition is also within the support. This situation is included in the description of the composition as “on” the support.

The ratio by weight of the aqueous phase to the oily phase in the composition of the invention preferably ranges from 25/75 to 90/10, more preferably 30/70 to 70/30, better still 40/60 to 60/40 and even better still 50/50. Thus, the aqueous phase generally represents from 25 to 90% by weight, preferably from 30 to 70% by weight and better still from 40 to 60% by weight, with respect to the total weight of the composition.

According to a preferred embodiment of the invention, the composition exhibits a viscosity preferably of less than 150 mpa·s and more preferably of less than 100 mPa·s. This viscosity preferably ranges from 1 mpa·s to 100 mpa·s, measured at ambient temperature (approximately 25° C.) with a Rheomat RM 180 device, rotor 1.

Aqueous Phase

The aqueous phase of the impregnation composition used according to the invention comprises water and optionally any water-soluble or water-dispersible additive(s). The water used can be sterile demineralized water and/or a floral water, such as rose water, cornflower water, camomile water or lime water, and/or a natural thermal or mineral water, such as, for example: water from Vittel, water from the Vichy basin, water from Uriage, water from La Roche Posay, water from La Bourboule, water from Enghien-les-Bains, water from Saint Gervais-les-Bains, water from Néris-les-Bains, water from Allevard-les-Bains, water from Digne, water from Maizières, water from Neyrac-les-Bains, water from Lons-le-Saunier, water from Eaux Bonnes, water from Rochefort, water from Saint Christau, water from Les Fumades, water from Tercis-les-Bains or water from Avene. The aqueous phase can also comprise reconstituted thermal water, that is to say a water comprising trace elements, such as zinc, copper, magnesium, and the like, reconstituting the characteristics of a thermal water.

Mention may in particular be made, as water-soluble additives, of polyols, such as glycerol, and glycols, such as hexylene glycol, polyethylene glycols and polypropylene glycol. The polyols can be present in an amount ranging, for example, from 0 to 5% by weight, preferably from 0.01 to 5% by weight, better still from 0.05 to 3% by weight and even better still from 0.1 to 3% by weight, with respect to the total weight of the composition. According to a preferred embodiment of the invention, the composition comprises at least one polyol, preferably glycerol or hexylene glycol or their mixtures.

Mention may also be made, as water-soluble additives, of primary C₂-C₈ alcohols and in particular of ethanol. According to a specific embodiment of the invention, the composition is preferably virtually devoid of ethanol. The term “virtually devoid of ethanol” is understood here to mean a composition comprising less than 2% by weight and preferably less than 1% by weight of ethanol, with respect to the total weight of the composition.

Oily Phase

The constituents of the oily phase preferably have a melting point of less than 25° C. and the oily phase thus preferably has a melting point of less than 25° C., that is to say that it is liquid at ambient temperature (25° C.).

The oily phase may generally represent from, e.g., 10 to 75% by weight, preferably from 30 to 70% by weight and better still from 40 to 60% by weight, with respect to the total weight of the composition.

The oily phase of the composition according to the invention can be composed of one or more oils, it being possible for the latter to be mineral, vegetable or synthetic oils or also silicone oils. It can additionally comprise fat-soluble or fat-dispersible additives. However, it is preferably devoid of compounds having a melting point of greater than 25° C. and in particular of waxes.

According to a preferred embodiment of the invention, the oily phase comprises one or more oils chosen from hydrocarbon oils of mineral or synthetic origin and silicone oils. More particularly, the oily phase advantageously comprises one or more volatile oils chosen from volatile hydrocarbon oils of mineral or synthetic origin and volatile silicone oils.

The term “hydrocarbon oil” is understood to mean an oil which is formed essentially, indeed even is composed, of carbon and hydrogen atoms and optionally of oxygen or nitrogen atoms and which does not comprise a silicon or fluorine atom; it can comprise ester, ether, amine or amide groups.

The term “volatile oil” is understood to mean an oil capable of evaporating on contact with the skin in less than one hour, at ambient temperature and atmospheric pressure. The volatile oil is a volatile cosmetic oil, liquid at ambient temperature, having in particular a nonzero vapour pressure, at ambient temperature and atmospheric pressure, especially having a vapour pressure ranging from 0.13 Pa to 40 000 Pa (10⁻³ to 300 mm of Hg), preferably ranging from 1.3 Pa to 13 000 Pa (0.01 to 100 mm of Hg) and preferentially ranging from 1.3 Pa to 1300 Pa (0.01 to 10 mm of Hg).

Mention may be made, as volatile hydrocarbon oils of mineral or synthetic origin, of C₈-C₁₆ isoalkanes (also known as isoparaffins), such as isododecane, isodecane or isohexadecane, such as, for example, the isoalkanes sold under the Isopar trade names by Exxon Chemical or the oils sold under the Permethyl trade names by Presperse; and their mixtures.

Mention may be made, as non-volatile hydrocarbon oils of mineral or synthetic origin, of mineral oil (INCI name), liquid petrolatum, hydrogenated polyisobutene, such as Parleam® oil, and their mixtures.

The term “silicone oil” is understood to mean an oil comprising at least one silicon atom and in particular comprising Si-O groups. The silicone oil can be chosen from non-volatile silicone oils, volatile silicone oils and their mixtures.

The volatile silicone oils which can be used in the invention can be chosen in particular from silicone oils having a flash point ranging from 40° C. to 102° C., preferably having a flash point of greater than 55° C. and of less than or equal to 95° C. and preferably ranging from 65° C. to 95° C. Mention may be made, as volatile silicone oils, of linear or cyclic silicone oils having from 2 to 7 silicon atoms, these silicones optionally comprising alkyl or alkoxy groups having from 1 to 10 carbon atoms. Mention may in particular be made, as examples of volatile silicone oils, of cyclopolydimethylsiloxanes (INCI name: cyclomethicone), such as cyclopentasiloxane, cyclohexasiloxane, octamethylcyclotetrasiloxane, decamethylcyclopentasiloxane or dodecamethylcyclohexasiloxane; linear silicones, such as heptamethylhexyltrisiloxane, heptamethyloctyltrisiloxane, hexamethyldisiloxane, octamethyltrisiloxane, decamethyltetrasiloxane or dodecamethylpentasiloxane, and their mixtures.

The non-volatile silicone oils which can be used in the invention can be chosen from polydimethylsiloxanes (PDMS), and phenylated polymethylsiloxanes, such as phenyl trimethicones, phenyl dimethicones, phenyl(trimethylsiloxy)diphenylsiloxanes, diphenyl dimethicones, diphenyl(methyldiphenyl)trisiloxanes, (2-phenylethyl) trimethylsiloxysilicates, and polymethylphenylsiloxanes; polysiloxanes modified by fatty acids, fatty alcohols or polyoxyalkylenes, and their mixtures.

According to a specific embodiment of the invention, the oily phase comprises at least one volatile oil and in particular at least one isoalkane chosen from isododecane, isohexadecane and their mixtures. According to another preferred embodiment of the invention, the oily phase comprises at least one isoalkane and at least one volatile silicone oil.

According to another specific embodiment of the invention, the oily phase comprises at least one mineral oil, in particular a mineral oil and a non-volatile silicone oil (dimethicone).

Furthermore, the oily phase can additionally comprise one or more other volatile or non-volatile oils chosen from hydrocarbon oils of animal or vegetable origin, synthetic esters and ethers, fatty alcohols, fluorinated oils and their mixtures.

The term “fluorinated oil” is understood to mean an oil comprising at least one fluorine atom.

Mention may be made, for example, as oils which can be used in the composition of the invention, of:
hydrocarbon oils of animal origin, such as perhydrosqualene;
hydrocarbon oils of vegetable origin, such as liquid triglycerides of fatty acids comprising from 4 to 10 carbon atoms, such as triglycerides of heptanoic acid or octanoic acid or also, for example, sunflower, maize, soybean, cucumber, grape seed, sesame, hazelnut, apricot kernel, macadamia, arara, coriander, castor or avocado oils, triglycerides of caprylic/capric acids, such as those sold by Stéarineries Dubois or those sold under the names Miglyol 810, 812 and 818 by Dynamit Nobel, jojoba oil or shea butter oil;
synethetic esters and ethers, in particular esters and ethers of fatty acids, such as oils of formulae R¹COOR² and R¹OR² in which R¹ represents the residue of a fatty acid comprising from 8 to 29 carbon atoms and R² represents a linear or branched hydrocarbon chain comprising from 3 to 30 carbon atoms, such as, for example, purcellin oil, isononyl isononanoate, isopropyl myristate, isopropyl palmitate, 2-ethylhexyl palmitate, 2-octyldodecyl stearate, 2-octyldodecyl erucate or isostearyl isostearate; hydroxylated esters, such as isostearyl lactate, octyl hydroxystearate, octyldodecyl hydroxystearate, diisostearyl malate or triisocetyl citrate; heptanoates, octanoates or decanoates of fatty alcohols; polyol esters, such as propylene glycol dioctanoate, neopentyl glycol diheptanoate and diethylene glycol diisononanoate; and pentaerythritol esters, such as pentaerythrityl tetraisostearate.

The oils can optionally be composed solely of volatile oils. The packaging of the foam stack will then consequently be chosen in order to guarantee good leaktightness thereof.

Surfactants

The two-phase composition according to the invention can optionally comprise one or more surfactants in one or other of the phases, in particular when it is used as make-up-removing or cleansing composition, as the presence of a surfactant makes it possible both to obtain good removal of make-up compositions and in particular-mascaras and also to have an absence of greasy feeling during make-up removal. However, the composition of the invention may also be devoid of surfactant and, if it comprises it, the amount of surfactant should be such that the composition remains, on standing, in the form of two separate phases and not in the form of an emulsion.

Thus, the amount of surfactant(s), as active material, preferably is in an amount such that the two phases remain separate on standing and do not mix with one another to form an emulsion. This amount should generally be less than or equal to 1.5% by weight, with respect to the total weight of the composition, depending on the surfactant. It can range, for example, from 0.001 to 1.5% by weight, preferably from 0.002 to 1% by weight and better still from 0.01 to 0.5% by weight, with respect to the total weight of the composition.

The surfactants can be chosen from nonionic, anionic, zwitterionic or amphoteric surfactants and their mixtures. According to a preferred embodiment of the invention, the surfactant, if it is present, is preferably nonionic.

Those which are particularly preferred among nonionic surface-active agents are:
polyoxyethylenated sorbitol fatty esters, such as the product sold under the name Tween 20 by ICI,
polyoxyethylenated fatty alcohols, such as the product sold under the name Remcopal 21912 AL by Gerland,
polyoxyethylenated alkylphenols, such as the product sold under then name Triton X 100 by Röhm & Haas,
condensates of ethylene oxide and of propylene oxide, such as those sold under the Synperonic PE names by ICI and in particular those referenced L 31, L 64, F 38, dispersible F 88, L 92, P 103, F 108 and F 127 (CTFA name: Poloxamer),
alkylpolyglycosides, such as those of following general formula (I):
   R-O-(G)ₓ (I)
   in which R represents a saturated or unsaturated and linear or branched alkyl radical comprising from 6 to 30 carbon atoms, G represents a reduced sugar comprising from 5 to 6 carbon atoms and x denotes a value ranging from 1 to 15. Mention may be made, as alkylpolyglucosides, for example, of decyl glucoside (Alkyl-C9/C11-polyglucoside (1.4)), such as the product sold under the name Mydol 10 by Kao Chemicals, the product sold under the names Plantaren 2000 UP and Plantacare 2000 UP by Cognis and the product sold under the name Oramix NS 10 by Seppic; caprylyl/capryl glucoside, such as the product sold under the name Oramix CG 110 by Seppic or under the name Lutensol GD 70 by BASF; lauryl glucoside, such as the products sold under the names Plantaren 1200 N and Plantacare 1200 by Cognis; and coco glucoside, such as the product sold under the name Plantacare 818/UP by Cognis; and their mixtures,
dimethicone copolyols or the mixtures comprising them, such as the product sold under the name 5225C by Dow Corning,
and their mixtures.

Mention may in particular be made, among anionic surface-active agents, of:
alkyl sulphates, alkyl ether sulphates and their salts, in particular their sodium salts, such as the Sodium Laureth Sulphate/Magnesium Laureth Sulphate/Sodium Laureth-8 Sulphate/Magnesium Laureth-8 Sulphate mixture sold under the name Texapon ASV by Cognis; sodium lauryl ether sulphate (CTFA name: sodium laureth sulfate), such as that sold under the names Texapon N 40 and Texapon AOS 225 UP by Cognis; ammonium lauryl sulphate (CTFA name: ammonium lauryl sulfate), such as the product sold by Huntsman under the name Empicol AL 30FL; ammonium alkyl (C12-C14) ether (9 EO) sulphate, sold under the name Rhodapex AB/20 by Rhodia Chimie; or triethanolamine lauryl sulphate (CTFA name: TEA-lauryl sulfate), such as the product sold by Huntsman under the name Empicol TL40 FL or that sold by Cognis under the name Texapon T42;
alkyl sulphoacetates, such as that sold under the name Lathanol LAL by Stepan;
alkyl sulphosuccinates, for example the oxyethylenated (3 EO) lauryl alcohol (C12/C14 70/30) monosulphosuccinate sold under the names Setacin 103 Special or Rewopol SB-FA 30 K 4 by Witco, the disodium salt of a hemisulphosuccinate of C₁₂-C₁₄ alcohols sold under the name Setacin F Special Paste by Zschimmer Schwarz, the oxyethylenated (2 EO) disodium oleamidosulphosuccinate sold under the name Standapol SH 135 by Cognis, the oxyethylenated (5 EO) lauramide monosulphosuccinate sold under the name Lebon A-5000 by Sanyo, the disodium salt of oxyethylenated (10 EO) lauryl citrate monosulphosuccinate sold under the name Rewopol SB CS 50 by Witco or the disodium salt of ricinoleic acid monoethanolamide monosulphosuccinate sold under the name Rewoderm S 1333 by Witco;
polypeptides which are obtained, for example, by condensation of a fatty chain with cereal amino acids and in particular wheat and oat amino acids, such as, for example, the potassium salt of lauroyl hydrolysed wheat protein sold under the name Aminofoam W OR by Croda, the triethanolamine salt of cocoyl hydrolysed soy protein sold under the name May-Tein SY by Maybrook, the sodium salt of lauroyl oat amino acids sold under the name Proteol Oat by Seppic, the hydrolysate of collagen grafted to coconut fatty acid sold under the name Geliderm 3000 by Deutsche Gelatine or the soy proteins acylated with hydrogenated coconut acids sold under the name Proteol VS 22 by Seppic;
amino acid derivatives, for example sarcosinates and in particular acylsarcosinates, such as sodium lauroyl sarcosinate, sold under then name Sarkosyl NL 97 by Ciba or sold under the name Oramix L 30 by Seppic, sodium myristoyl sarcosinate, sold under the name Nikkol Sarcosinate MN by Nikkol, or sodium palmitoyl sarcosinate, sold under the name Nikkol sarcosinate PN by Nikkol; alaninates, such as sodium N-lauroyl-N-methylamidopropionate, sold under the name Sodium Nikkol Alaninate LN 30 by Nikkol or sold under the name Alanone ALE by Kawaken, and triethanolamine N-lauroyl-N-methylalanine, sold under the name Alanone ALTA by Kawaken; N-acylglutamates, such as triethanolamine monococoyl glutamate, sold under the name Acylglutamate CT-12 by Ajinomoto, and triethanolamine lauroyl glutamate, sold under the name Acylglutamate LT-12 by Ajinomoto; aspartates, such as a mixture of triethanolamine N-lauroyl aspartate and of triethanolamine N-myristoyl aspartate sold under the name Asparack LM-TS2 by Mitsubishi; or glycine derivatives, such as sodium N-cocoyl glycinate and potassium N-cocoyl glycinate, for example the products sold under the names Amilite GCS-12 and Amilite GCK-12 by Ajinomoto;
sulphonates, for example α-olefin sulphonates, such as the sodium α-olefin (C14-16) sulphonate sold under the name Bio-Terge AS-40 by Stepan, sold under the names Witconate AOS Protégé and Sulframine AOS PH 12 by Witco or sold under the name Bio-Terge AS-40 CG by Stepan or the sodium secondary olefin sulphonate sold under the name Hostapur SAS 30 by Clariant; or linear alkylarylsulphonates, such as the sodium xylenesulphonate sold under the names Manrosol SXS30, Manrosol SXS40 or Manrosol SXS93 by Manro;
isethionates, in particular acyl isethionates, such as sodium cocoyl isethionate, for example the product sold under the name Jordapon CI P by Jordan.

Mention may in particular be made, among amphoteric or zwitterionic surfactants, of:
alkylamido alkylamine derivatives, such as N-disodium N-cocoyl-N-carboxymethoxyethyl-N-(carboxymethyl)-ethylenediamine (CTFA name: disodium cocoamphodiacetate), sold as a saline aqueous solution under the name Miranol C2M Conc. NP by Rhodia Chimie; N-sodium N-cocoyl-amidoéthyl, N-hydroxyéthyl glycinate CTFA name: sodium cocoamphoacetate); and the mixture of coconut acid ethanolamides (CTFA name: cocamide DEA);
betaines, such as, for example, coco betaine, such as the product sold under the name Dehyton AB-30 by Cognis, lauryl betaine, such as the product sold under the name Genagen KB by Clariant, oxyethylenated (10 EO) lauryl betaine, such as the product sold under the name Lauryl Ether (10 EO) Betaine by Shin Nihon Rica, or oxyethylenated (10 EO) stearyl betaine, such as the product sold under the name Stearyl Ether (10 EO) Betaine by Shin Nihon Rica;
alkyl amidopropyl betaines and their derivatives, such as, for example cocamidopropyl betaine, sold under the name Lebon 2000 HG by Sanyo or sold under the name Empigen BB by Albright & Wilson or sold under the names Tegobetaine by Goldschmidt, lauramidopropyl betaine, sold under the name Rewoteric AMB12P by Witco ;
imidazoline derivatives, such as the product sold under the name Chimexane HD by Chimex; and
their mixtures.
   Phase Separation Agent

The two-phase composition can additionally comprise one or more agents which promote phase separation into two phases after agitation in order to reduce the phase-separation time. Mention may be made, as phase-separation agents, for example, of alkyldimethylbenzylammonium chlorides, as disclosed in the document EP-A-0 603 080, esters of dimethylcarboxylic acid and of alkanediol, sodium bicarbonate, alkoxylated alkylglucosides possessing quaternary ammonium, as disclosed in the document EP-A-0 847 746, PVP copolymers, as disclosed in the document EP-A-0 996 408, or a mixture of polydextrose and sucrose, as disclosed in the document U.S. Pat. No. 6,727,209. These agents can be present in an amount ranging, for example, from 0.005 to 5%, preferably from 0.01 to 4% and better still from 0.5 to 4% of the total weight of the composition.

Adjuvants

In addition to those indicated above, the two-phase composition used in the article according to the invention can also comprise, e.g., cosmetic adjuvants or additives which will be found in one or other phase, depending on their hydrophilic or lipophilic nature, such as, for example, fragrances, colorants, preservatives, bactericides, softening agents, buffers, humectants, UV screening agents (or sunscreens), electrolytes, such as sodium chloride, as indicated above, or a pH adjuster (for example, citric acid or sodium hydroxide), and their mixtures.

Use may be made, as preservatives, of any preservative commonly used in the fields under consideration, such as, for example, parabens, chlorohexidine gluconate, polyhexamethylene biguanide hydrochloride (CTFA name: polyaminopropyl biguanide) and their mixtures. According to a preferred embodiment of the invention, the composition comprises polyhexamethylene biguanide hydrochloride, alone or as a mixture with other preservatives.

Use may be made, as bactericide, for example, of a glyceryl mono (C₃-C₉) alkyl or mono (C₃-C₉) alkenyl ether, the manufacture of which is described in the literature, in particular in E. Baer and H. O. L. Fischer, J. Biol. Chem.,1941, 140, p.397. Use is preferably made, among these glyceryl mono(C₃-C₉)alkyl or mono (C₃-C₉) alkenyl ethers, of 3-[(2-ethylhexyl)oxy]-1,2-propanediol, 3-[(heptyl)oxy]-1,2-propanediol, 3-[(octyl)oxy]-1,2-propanediol and 3-[(allyl)oxy]-1,2-propanediol. A glyceryl mono(C₃-Cg)alkyl ether which is more particularly preferred according to the present invention is 3-[(2-ethylhexyl)oxy]-1,2-propanediol, sold by Schulke & Mayr GmbH under the trade name Sensiva SC 50 (INCI name: ethylhexylglycerin).

Mention may in particular be made, among the softening agents, of allantoin, bisabolol, plankton and some plant extracts, such as rose extracts and melilot extracts.

The active principle or principles which can be present depend on the final purpose of the composition. Mention may be made, as active principles which can be used in the composition of the invention, for example, of enzymes (for example, lactoperoxidase, lipase, protease, phospholipase or cellulases); moisturizing agents, such as protein hydrolysates; sodium hyaluronate; anti-inflammatories; procyanidol oligomers; vitamins, such as vitamin A (retinol), vitamin E (tocopherol), vitamin C (ascorbic acid), vitamin B5 (panthenol), vitamin B3 (niacinamide), the derivatives of these vitamins (in particular esters) and their mixtures; urea; caffeine; depigmenting agents, such as kojic acid, hydroquinone and caffeic acid; salicylic acid and its derivatives; α-hydroxy acids, such as lactic acid and glycolic acid, and their derivatives; antibacterial active principles, such as 2,4,4′-trichloro-2′-hydroxydiphenyl ether (or triclosan), 3,4,4′-trichlorocarbanilide (or triclocarban) and the acids indicated above and in particular salicylic acid and its derivatives; essential oils; and their mixtures; and any active principle appropriate for the final purpose of the composition.

The lipophilic adjuvants are dissolved directly in the oils, whereas the hydrophilic adjuvants are added to the aqueous phase or are dispersed in the composition using the surfactants present.

Stability Test on the Articles 1) Composition used

For the comparative stability tests on the stacks of articles, use was made of the following two-phase composition, where the percentages are percentages by weight:

| Oily phase | |
|---|---|
| Mineral oil | 50% |
| Dimethicone | 50% |
| Aqueous phase | |
| Hexylene glycol | 0.5% |
| PEG-60 Hydrogenated Castor Oil | 0.2% |
| Decyl glucoside | 0.2% |
| Fragrance | 0.017% |
| Disodium EDTA (chelating agent) | 0.2% |
| Preservative | 1.8% |
| Sodium chloride | 0.5% |
| Demineralized water | 96.583% |

Procedure: The constituents of the oily phase, on the one hand, and those of the aqueous phase, on the other hand, are mixed. The two phases are then mixed. A composition is obtained which, on standing, comprises a separate aqueous phase and a separate oily phase. On agitating, the two phases give an emulsion which again separates on standing to give two phases.

2) Technique for Determining the Sedimentation of the Oily and Aqueous Fractions within a Stack of Articles after a Storage Time

(To be used when the oily phase does not comprise volatile oil)

Before carrying out this test, it is necessary, for a given support and a given composition (1) to confirm that the support does not comprise residual water or residual solvent after having been left at 45° C. for 72 hours; (2) to confirm that all the amount of water is evaporated when this support, impregnated with 500% of aqueous phase, is stored at 45° C. for 72 hours and, if not, to deduce therefrom the residual fraction of aqueous phase on the support, and (3) to confirm that all the oily phase is retained on the support when this support, impregnated with 500% of oily phase, is stored at 45° C. for 72 hours.

In the case of the supports used in the examples of the invention and in the comparative example, it was found (1) that there was no loss in weight of the support after 72 hours at 45° C., which means that these supports comprise neither residual water nor residual solvent, (2) that there remained a residual amount of aqueous phase of the support impregnated with aqueous phase, which amount is taken into account in the calculation presented below, and (3) that there was no loss of oily phase, which is logical since the above impregnation composition does not comprise volatile oil.

In order to determine the aqueous and oily fractions in the articles, the article is weighed, is placed in an oven at 45° C. for 24 hours and is reweighed on its removal from the oven.

In the case of the foams, each impregnated support is taken separately. In the case of the nonwoven discs, use for the comparative example, these nonwovens being lighter, they are much more numerous in stack than the discs of polyurethane foam for a box of identical size. Thus, for the weighings and the sedimentation calculations, the nonwoven discs, to the number of 60 in each box, are weighed in groups of 5 (that is to say, 12 weighings for a stack of 60 nonwoven discs), whereas the discs of polyurethane foam, to the number of 15 in each box, are weighed individually (that is to say, 15 weighings for a stack of 15 foam discs).

The impregnated aqueous fraction is calculated in the following way:
W aqueous phase=W impregnated (before moving to the oven)−W impregnated (after 24 h at 45° C.)+W residual fraction of aqueous phase on the support

The impregnated oily fraction is calculated in the following way:
W oily phase=W impregnated (after 24 h at 45° C.)−W nonimpregnated−W residual fraction of aqueous phase on the support
W impregnated is the weight of the support impregnated with the composition
W nonimpregnated is the weight of the support before impregnation
W residual fraction of aqueous phase on the support is the weight of aqueous phase remaining trapped in the foam (=residual amounts of aqueous phase of the support impregnated with aqueous phase)

This method thus makes it possible to precisely determine the amounts of aqueous and oily phases present on a support, such as the hydrophilic polyurethane foam used in the example according to the example below and such as the nonwoven used in the comparative example below.

Before using this method for another support, it is recommended to confirm, by a simple oven test, its behaviour in the presence of water, of wool and of the mixture. As indicated above, in the case of the foam given as example, a non-volatile water fraction will have to be subtracted or added.

### 3) Comparative Example with a Nonwoven Support

60 round sheets (diameter=57 mm) of 100% cotton nonwoven (grammage=100 ±5 g/m²) were impregnated with the two-phase composition indicated above, at a degree of impregnation of 500% (5 g of make-up remover per 1 g of nonwoven), that is to say by introducing 250% of aqueous phase and 250% of oily phase onto each nonwoven sheet (2.5 g of aqueous phase and 2.5 g of oily phase per 1 g of nonwoven).

For this nonwoven support, there is no residual water, that is to say no residual amount of aqueous phase of the support impregnated with aqueous phase, which would be necessary to take into account in the calculation.

The height of the stack of dry nonwoven disks is 4.8 cm. The sheets are packaged in a plastic box typical of a box of make-up-removing pads.

After a storage time of two weeks, the new istribution of the liquid in the stack of nonwoven isks was measured (Table 1).

TABLE 1

| | Weight of | Results at | | Results after | |
|---|---|---|---|---|---|
| | support | time zero | | 15 days | |
| | Weight | Weight | Weight | Weight | Weight |
| | (W) of | (W) of | (W) of | (W) of | (W) of |
| | dry | aqueous | oily | aqueous | oily |
| Nonwoven | nonwoven | phase | phase | phase | phase |
| sheet | (g) | (g) | (g) | (g) | (g) |
| 5 from | 1.25 | 3.16 | 3.19 | 2.75 | 3.26 |
| the top | | | | | |
| 5 following | 1.32 | 3.3 | 3.31 | 3.12 | 3.33 |
| 5 following | 1.33 | 3.32 | 3.34 | 3.2 | 3.36 |
| 5 following | 1.38 | 3.47 | 3.43 | 3.33 | 3.44 |
| 5 following | 1.39 | 3.49 | 3.49 | 3.31 | 3.51 |
| 5 following | 1.36 | 3.42 | 3.48 | 3.26 | 3.47 |
| 5 following | 1.35 | 3.38 | 3.49 | 3.28 | 3.51 |
| 5 following | 1.31 | 3.28 | 3.28 | 3.32 | 3.12 |
| 5 following | 1.34 | 3.35 | 3.36 | 3.46 | 3.29 |
| 5 following | 1.34 | 3.35 | 3.35 | 3.51 | 3.26 |
| 5 following | 1.26 | 3.17 | 3.20 | 3.32 | 3.12 |
| 5 from the | 1.35 | 3.38 | 3.39 | 3.60 | 3.30 |
| bottom | | | | | |

This table shows that, after 15 days, the proportions of aqueous phase and of oily phase are no longer the same in the various articles depending on their position in the stack of articles and that the articles at the bottom of the stack comprise much more aqueous phase than those at the top of the stack.

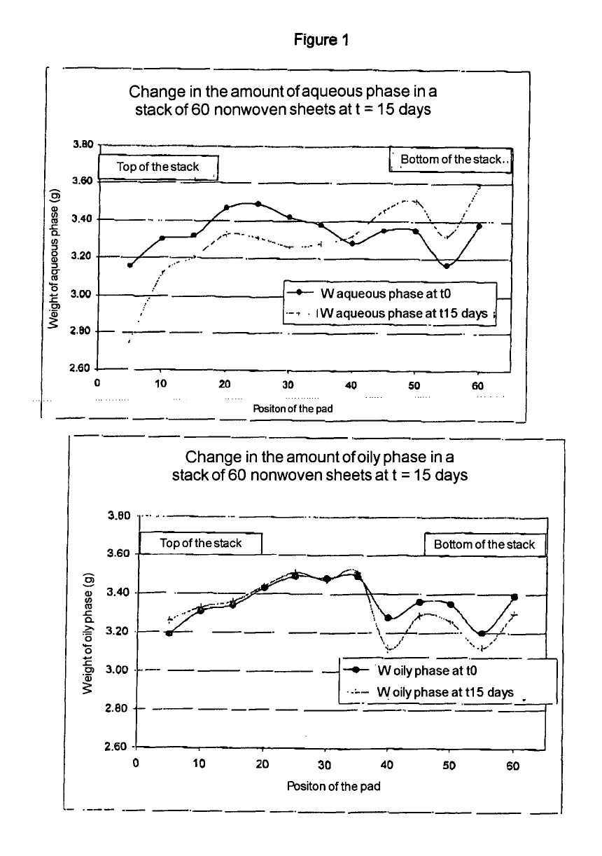

The phenomenon observed for each phase is illustrated in Graphs 1 and 2 of FIG. 1.

### 4) Example 1 According to the Invention

15 round supports made of hydrophilic polyurethane foam (Martini, thickness of 3 mm, diameter of 50 mm) were impregnated to 500% with the composition indicated above, i.e. 250% of aqueous phase and 250% of oily phase. The height of this stack of 15 dry foam discs is 4.8 cm. The foams are packaged in a plastic box typical of a box of make-up-removing pads (the same as in the above comparison example).

After storing for 2 months, the amounts of aqueous phase and of oily phase present in each foam are determined. The results are represented in Table 2.

TABLE 2

| | Weight of | Results at time zero | | Results after 2 months | |
|---|---|---|---|---|---|
| | support | Weight | Weight | Weight | Weight |
| | Weight | (W) of | (W) of | (W) of | (W) of |
| | (W) of | aqueous | oily | aqueous | oily |
| | dry foam | phase | phase | phase | phase |
| Foam | (g) | (g) | (g) | (g) | (g) |
| Top | 0.56 | 1.45 | 1.37 | 1.43 | 1.44 |
| Following | 0.58 | 1.51 | 1.41 | 1.33 | 1.35 |
| Following | 0.59 | 1.53 | 1.43 | 1.51 | 1.48 |
| Following | 0.55 | 1.41 | 1.36 | 1.37 | 1.39 |
| Following | 0.58 | 1.48 | 1.45 | 1.46 | 1.43 |
| Following | 0.60 | 1.51 | 1.49 | 1.38 | 1.46 |
| Following | 0.62 | 1.56 | 1.54 | 1.61 | 1.62 |
| Following | 0.59 | 1.50 | 1.46 | 1.40 | 1.42 |
| Following | 0.58 | 1.46 | 1.46 | 1.38 | 1.41 |
| Following | 0.58 | 1.51 | 1.41 | 1.40 | 1.42 |
| Following | 0.59 | 1.49 | 1.49 | 1.39 | 1.48 |
| Following | 0.63 | 1.59 | 1.59 | 1.59 | 1.62 |
| Following | 0.58 | 1.46 | 1.49 | 1.37 | 1.44 |
| Following | 0.51 | 1.29 | 1.25 | 1.36 | 1.27 |
| Bottom | 0.60 | 1.51 | 1.49 | 1.59 | 1.44 |

This table shows that, after 2 months, the proportions of aqueous phase and of oily phase remain virtually the same in the articles of the stack and that the articles at the bottom of the stack comprise an amount of aqueous phase which does not vary by much in comparison with the original amounts. For the article at the top, the loss of aqueous phase is 1.38%, whereas it is 12.97% on the nonwoven support, and, for the bottommost article, the increase in aqueous phase is 5.3%, whereas it is 6.5% for the nonwoven support.

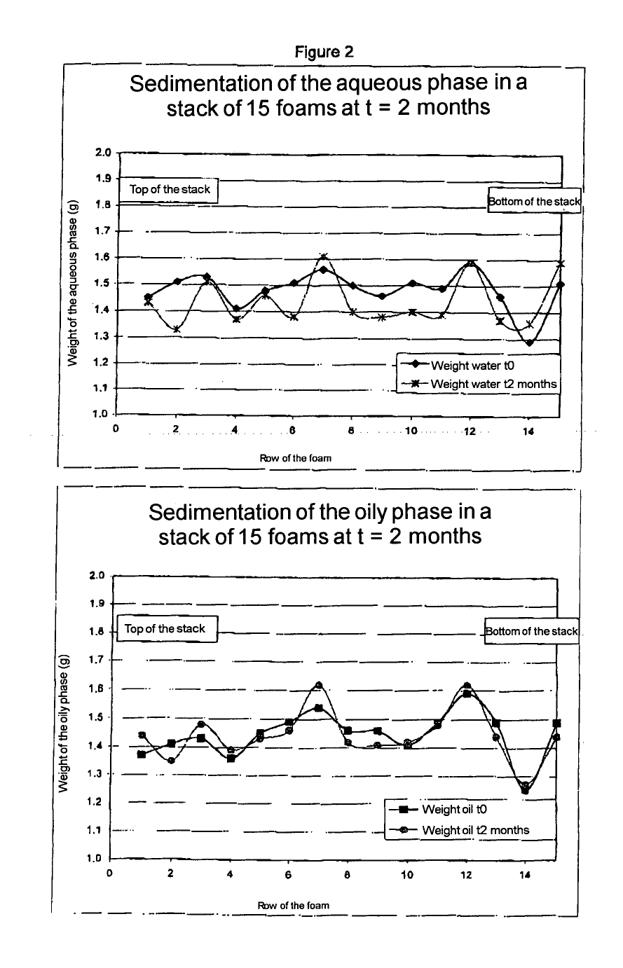

The phenomenon observed for each phase is illustrated in Graphs 3 and 4 of FIG. 2.

### Examples 2 to 5 According to the Invention

The examples below of compositions according to the invention are given by way of illustration and without their limiting nature. The names are chemical names or CTFA names. The amounts are given therein as % by weight, unless otherwise mentioned.

### Example 2

### Make-up-removing Composition

| Oily phase | |
|---|---|
| Cyclopentasiloxane | 14% |
| Isododecane | 15% |
| Isopropyl palmitate | 19.6% |
| Fragrance | 0.2% |
| Ethylhexylglycerin (Sensiva SC 50) | 1.2% |
| Aqueous phase | |
| Glycerol | 20% |
| Dipropylene glycol | 12% |
| Triethanolamine (neutralizing agent) | 0.07% |
| Methylparaben | 0.2% |
| Sodium chloride | 0.5% |
| Disodium EDTA (chelating agent) | 0.08% |
| Demineralized water | 17.15% |

Procedure: The constituents of the oily phase, on the one hand, and those of the aqueous phase, on the other hand, are mixed. The two phases are then mixed.

A composition is obtained which, on standing, comprises a separate aqueous phase and a separate oily phase. This composition is impregnated on a support of Martini hydrophilic polyurethane foam as in Example 1. The article obtained can be used for removing make-up from the skin.

### Example 3

| Oily phase | |
|---|---|
| Cyclopentasiloxane | 35% |
| Isododecane | 7.5% |
| Isohexadecane | 7.5% |
| Aqueous phase | |
| Glycerol | 1.5% |
| Polyhexamethylene biguanide hydrochloride | 0.35% |
| Sodium bicarbonate | 1% |
| Demineralized water | 47.15% |

Procedure: The constituents of the oily phase, on the one hand, and those of the aqueous phase, on the other hand, are mixed. The two phases are then agitated.

A composition is obtained which, on standing, comprises a separate aqueous phase and a separate oily phase. This composition is impregnated on a foam support like that of Example 1. The article obtained can be used for removing make-up from the skin.

### Example 4

| Oily phase | |
|---|---|
| Cyclopentasiloxane | 27% |
| Isohexadecane | 19% |
| Ester of 7,7-dimethyloctanoic acid and | 2% |
| of 1,2-propanediol | |
| Aqueous phase | |
| Hexylene glycol | 0.27% |
| Fragrance | 0.01% |
| Decyl glucoside (55% aqueous solution) | 0.06% |
| Monopotassium phosphate | 0.15% |
| Dipotassium phosphate | 0.03% |
| Polyhexamethylene biguanide hydrochloride | 0.08% |
| (20% aqueous solution) | |
| Sodium chloride | 0.26% |
| Water | 49.14% |

Procedure: The constituents of the oily phase, on the one hand, and those of the aqueous phase, on the other hand, are mixed. The two phases are then agitated.

A composition is obtained which, on standing, comprises a separate aqueous phase and a separate oily phase. This composition is impregnated on a foam support like that of Example 1. The article obtained can be used for removing make-up from the skin.

### Example 5

| | |
|---|---|
| Oily phase | |
| Cyclomethicone | 28% |
| Isohexadecane | 19% |
| Aqueous phase | |
| Poloxamer 184 | 0.05% |
| Phosphate buffer | 0.15% |
| Sodium chloride | 0.6% |
| Preservatives | q.s. |
| Colorants | q.s. |
| Demineralized water | q.s. for 100% |

Procedure: The constituents of the oily phase, on the one hand, and those of the aqueous phase, on the other hand, are mixed. The two phases are then agitated.

A composition is obtained which, on standing, comprises a separate aqueous phase and a separate oily phase. This composition is impregnated on a foam support like that of Example 1. The article obtained can be used for removing make-up from the skin.

The above written description of the invention provides a manner and process of making and using it such that any person skilled in this art is enabled to make and use the same, this enablement being provided in particular for the subject matter of the appended claims, which make up a part of the original description and including an article comprising:

(A) a water-insoluble hydrophilic polyurethane foam support, and, impregnated on the support,

(B) a composition comprising separate, non-emulsified aqueous and oily phases, said aqueous and oily phases being present in a ratio by weight ranging from 25/75 to 90/10 (aqueous/oily), the oily phase having a melting point of less than 25° C., as well as an article comprising (A) a water-insoluble support composed of a hydrophilic polyurethane foam and (B) a cosmetic composition impregnated on the support and composed of a separate aqueous phase and a separate oily phase in a ratio by weight ranging from 25/75 to 90/10, the constituents of the oily phase having a melting point of less than 25° C.

As used above, the phrases “selected from the group consisting of,” “chosen from,” and the like include mixtures of the specified materials. Terms such as “contain(s)” and the like as used herein are open terms meaning ‘including at least’ unless otherwise specifically noted.

All references, patents, applications, tests, standards, documents, publications, brochures, texts, articles, etc. mentioned herein are incorporated herein by reference. Where a numerical limit or range is stated, the endpoints are included. Also, all values and subranges within a numerical limit or range are specifically included as if explicitly written out.

The above description is presented to enable a person skilled in the art to make and use the invention, and is provided in the context of a particular application and its requirements. Various modifications to the preferred embodiments will be readily apparent to those skilled in the art, and the generic principles defined herein may be applied to other embodiments and applications without departing from the spirit and scope of the invention. Thus, this invention is not intended to be limited to the embodiments shown, but is to be accorded the widest scope consistent with the principles and features disclosed herein.

1. An article comprising:
(A) a water-insoluble hydrophilic polyurethane foam support, and, impregnated on the support,
(B) a composition comprising separate, non-emulsified aqueous and oily phases, said aqueous and oily phases being present in a ratio by weight ranging from 25/75 to 90/10 (aqueous phase/oily phase), the oily phase having a melting point of less than 25° C.

2. The article according to claim 1, wherein the support is an open cell polyurethane foam.

3. The article according to claim 1, wherein the foam is elastic.

4. The article according to claim 1, wherein the foam has a maximum water-absorbing power of 0.4 to 3.5 g/cm³ and a water-retaining power of 0.07 to 2 g/cm³.

5. The article according to claim 1, wherein the foam has a thickness of 0.5 to 10 mm.

6. The article according to claim 1, wherein the foam has a surface area of 0.005 m² to 0.1 m₂.

7. The article according to claim 1, wherein the degree of impregnation of the composition on the support is 10 to 1500% by weight, with respect to the weight of support.

8. The article according to claim 1, wherein the ratio by weight of the aqueous phase to the oily phase in the composition is 30/70 to 70/30.

9. The article according to claim 1, wherein the composition further comprises one or more surfactant(s) in an amount of 0.001 to 1.5% by weight, with respect to the total weight of the composition.

10. The article according to claim 1, wherein the composition exhibits a viscosity of less than 150 mpa·s.

11. The article according to claim 1, wherein it constitutes a wipe, compress, pad or sponge.

12. The article according to claim 1, wherein it constitutes an article for cleansing and/or removing make-up from the skin, lips and/or eyes.

13. A process for cleansing and/or removing make-up from the skin, lips and/or eyes, which comprises passing over the skin, lips and/or eyes an article according to claim 1.
